(19) United States
(12) Patent Application Publication
(10) Pub. No.: US 2025/0032176 A1
(43) Pub. Date: Jan. 30, 2025

(54) NEUROMODULATION CATHETER INCLUDING MULTIPLE HELICAL THERAPEUTIC ELEMENTS

(71) Applicant: Medtronic Ireland Manufacturing Unlimited Company, Dublin (IE)

(72) Inventors: Jaclyn N. Kawwas, Philadelphia, PA (US); Manoj Kumar Singh, Santa Rosa, CA (US); Carlos H. Lima, Santa Rosa, CA (US)

(21) Appl. No.: 18/716,707

(22) PCT Filed: Dec. 9, 2022
(86) PCT No.: PCT/EP2022/085140

§ 371 Date: Jun. 5, 2024

Related U.S. Application Data
(60) Provisional application No. 63/292,584, filed on Dec. 22, 2021.

(51) Int. Cl. A61B 18/14; A61B 18/00

(52) U.S. Cl. CPC A61B 18/1492; A61B 2018/0016; A61B 2018/00202; A61B 2018/00214; A61B 2018/00404; A61B 2018/00434; A61B 2018/1475

(57) ABSTRACT

A system may include a catheter that includes an elongated member configured to navigate through a vasculature of a patient to a target treatment site and a plurality of expandable members arranged at a distal portion of the member. Each expandable member may include a corresponding two or more therapeutic elements arranged along a length of the expandable member. Each expandable member of the plurality of expandable members may be configured to assume a delivery configuration and a radially expanded configuration. Each expandable member may be configured to position the corresponding two or more therapeutic elements near a vessel wall so that the therapeutic elements are spaced around a perimeter of the vessel wall. In some examples, distal portions of the plurality of expandable members may be connected to each other at a distal tip of the catheter.

### TECHNICAL FIELD

The present technology is related to catheters configured to deliver neuromodulation therapy.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

### SUMMARY

The present disclosure describes devices, systems, and methods for neuromodulation, such as renal neuromodulation. In other examples, systems, devices, and methods described herein may be useful for neuromodulation within a body lumen other than a vessel, for extravascular neuromodulation, for non-renal-nerve neuromodulation, and/or for use in therapies other than neuromodulation.

In some examples, the disclosure describes a system including a catheter that includes an elongated member configured to navigate through a vasculature of a patient to a target treatment site and a plurality of expandable members arranged at a distal portion of the member. Each curved member may include a corresponding two or more therapeutic elements arranged along a length of the expandable member. Each expandable member of the plurality of expandable members may be configured to assume a delivery configuration and a radially expanded helical configuration. Each expandable member may be configured to position the corresponding two or more therapeutic elements near a vessel wall so that the therapeutic elements are spaced around a perimeter of the vessel wall. Distal portions of the plurality of expandable members may be connected to each other at a distal tip of the catheter.

In some examples, the disclosure describes a method that includes navigating a catheter through vasculature of a patient to a target treatment site. The catheter includes an elongated member and a plurality of expandable members arranged at a distal portion of the elongated member. Each expandable member may include a corresponding two or more therapeutic elements arranged along a length of the expandable member. Distal portions of the plurality of expandable members may be connected to each other at a distal tip of the catheter. The method also includes expanding each expandable member of the plurality of expandable members from a delivery configuration to radially expanded helical configuration. When the plurality of expandable members are in the radially expanded helical configuration, the corresponding two or more therapeutic elements of each expandable member are positioned near a vessel wall so that the therapeutic elements are spaced around a perimeter of the vessel wall. The method further includes delivering treatment to the vessel wall using the one or more therapeutic elements.

Further disclosed herein is a system that includes a catheter that includes an elongated member configured to navigate through a vasculature of a patient to a target treatment site and a plurality of expandable members arranged at a distal portion of the member, wherein each expandable member may include a corresponding two or more therapeutic elements arranged along a length of the expandable member, wherein each expandable member of the plurality of expandable members may be configured to assume a delivery configuration and a radially expanded configuration, wherein each expandable member may be configured to position the corresponding two or more therapeutic elements near a vessel wall so that the therapeutic elements are spaced around a perimeter of the vessel wall, and wherein, in some examples, distal portions of the plurality of expandable members may be connected to each other at a distal tip of the catheter.

The details of one or more examples are set forth in the accompanying drawings and the description below. Other features, objects, and advantages will be apparent from the description and drawings, and from the claims.

### BRIEF DESCRIPTION OF THE DRAWINGS

Reference is made to the attached drawings, wherein elements have the same reference numeral designations represent similar elements throughout.

FIG. 1 is a partial schematic illustration of a neuromodulation system configured in accordance with some examples of the present disclosure.

FIG. 2 illustrates a technique for accessing a renal artery and modulating renal nerves with the system of FIG. 1 in accordance with some examples of the present disclosure.

FIG. 3 is a conceptual illustration of an example sympathetic nervous system (SNS) illustrating how the brain communicated with the body via the SNS.

FIG. 4 is an enlarged anatomic view of nerves innervating a left kidney to form the renal plexus surrounding the left renal artery.

FIG. 5 is an anatomic view of a human body depicting neural efferent and afferent communication between the brain and kidneys.

FIG. 6 is a conceptual view of a human body depicting neural efferent and afferent communication between the brain and kidneys.

FIG. 7 is an anatomic view of the arterial vasculature of a human.

FIG. 8 is an anatomic view of the venous vasculature of a human.

FIG. 9 is a conceptual diagram illustrating an example neuromodulation catheter that includes a distal portion of an elongated member positioned within a renal vessel, in accordance with some examples of the present disclosure.

FIG. 10 is a conceptual diagram illustrating a cross-section view of another example neuromodulation catheter with a guide wire inside an inner lumen of an expandable member, in accordance with some examples of the present disclosure.

FIG. 11 is a conceptual diagram illustrating an example guide sheath including an extended sheath tip, in accordance with some examples of the present disclosure.

FIG. 12 is a conceptual diagram illustrating another example neuromodulation catheter that includes a guide member, in accordance with some examples of the present disclosure.

FIG. 13 is a conceptual diagram illustrating another example neuromodulation catheter, which includes a plurality of expandable members, in accordance with some examples of the present disclosure.

FIG. 14 is a flow diagram illustrating an example technique for performing neuromodulation of renal nerves, in accordance with some examples of this disclosure.

### DETAILED DESCRIPTION

The present disclosure describes devices, systems, and method for neuromodulation, such as renal neuromodulation, using therapeutic elements, such as electrodes, cryogenic elements, or the like. Although renal neuromodulation is primarily described herein, devices, systems, and techniques described herein may be applied to other types of neuromodulation, such as neuromodulation performed on nerves other than the renal nerves, at sites other than within a renal vessel, or both. In general, the devices, systems, and techniques described herein may be used to perform neuromodulation from within any suitable anatomical lumen that has nerves adjacent to the anatomical lumen.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction towards the clinician or clinician's control device.

Neuromodulation, such as renal denervation, may be accomplished using one or more of a variety of treatment modalities, including radio frequency (RF) energy, microwave energy, ultrasound energy, heat, cryogenic cooling, a chemical agent, or the like. To perform intravascular neuromodulation, a neuromodulation catheter may be delivered to a vessel, such as a renal artery, of a patient. The neuromodulation catheter may include one or more expandable members, each of which includes one or more therapeutic elements. The one or more therapeutic elements may be positioned in apposition to the vessel wall to transfer energy (e.g., RF energy, heat, cooling, etc.) to or from tissue surrounding the vessel wall.

In some examples, a clinician exchanges energy to the nerves located adjacent to the vessel in which the one or more expandable members is positioned using the one or more therapeutic elements to ablate at least some nerves adjacent to the vessel. The nerve locations with respect to the vessel may vary widely among patients, particularly for renal nerves around a renal vessel. To achieve successful therapy, it may be desired to treat as many nerves as possible.

In some examples, therapeutic elements may be carried by a helical structure, such as a helical catheter, and may be positioned spaced apart both circumferentially and longitudinally along the vessel wall. This may result in non-continuous lesion formation at locations approximately corresponding to the therapeutic element locations. Together, the non-continuous lesions may surround a perimeter (e.g., circumference) of the vessel, but there may be spaces between adjacent non-continuous lesions due to the longitudinal spacing between the therapeutic elements. While this may provide efficacious neuromodulation therapy for some patients, for other patients this lesion distribution may be less effective, e.g., due to where nerves approach the vessel wall. Less effective neuromodulation therapy may lead to additional treatments, e.g., after repositioning the therapeutic elements within the vessel or in follow-up operations. The additional treatments may increase total treatment time and increase overall cost of the procedure. Similarly, follow-up operations may increase an overall cost of patient treatment and may not be pursued by all patients who could benefit from it.

In accordance with techniques of this disclosure, a neuromodulation catheter may include one or more features configured to enable the creation of a substantially continuous circumferential lesions surrounding a vessel in which the neuromodulation catheter is positioned. The neuromodulation catheter may include an elongated member configured to be navigated through vasculature of a patient to a target treatment site and a plurality of expandable members arranged at a distal portion of the elongated member. For example, the neuromodulation catheter may include two expandable members. Each expandable member may include a corresponding one or more therapeutic elements arranged along the length of the expandable member. For instance, each expandable member may include two or three therapeutic elements arranged along the length of the expandable member.

Each expandable member of the plurality of expandable members may be configured to assume a delivery configuration in which the plurality of expandable members define a relatively smaller radial extent and a radially expanded configuration. In some examples, the radially expanded configuration may include a radially expanded helical configuration. In other examples, the radially expanded configuration may include a radially expanded curved configuration. In the radially expanded configuration, each expandable member is configured to position the corresponding one or more therapeutic elements carried by the respective expandable member near a vessel wall, e.g., in apposition to the vessel wall. The therapeutic elements may be positioned along each expandable member such that, when the plurality of expandable members are in the radially expanded configuration, the therapeutic elements are spaced around an inner perimeter (e.g., circumference) of the vessel wall. In this way, the neuromodulation catheter may enable formation of substantially circumferentially continuous lesions around the vessel wall. In some examples, the therapeutic elements may be positioned along each expandable member such that, when the plurality of expandable members are in the radially expanded configuration, at least part of each therapeutic element is intersected by a plane substantially orthogonal to a longitudinal axis of the expandable member.

Further, in some implementations, distal portions of the expandable members may be connected at a distal tip of the catheter. This may reduce extension of the expandable members in the radially expanded deployed configuration. In some implementations, the distal tip may further include a longitudinally extended shape that facilitates advancement of the catheter through vasculature to the target treatment site.

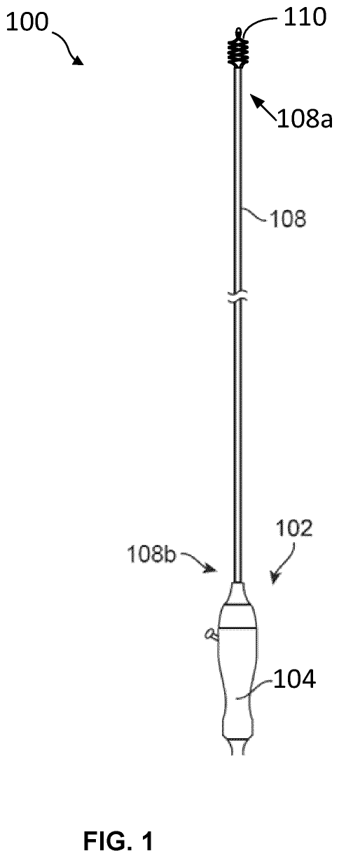

FIG. 1 is a partially schematic illustration of neuromodulation system 100 (“system 100”) configured in accordance with some examples of the present disclosure. As shown in FIG. 1, system 100 includes a neuromodulation catheter 102, which includes a handle 106 and an elongated member 108 attached to handle 106. Elongated member 108 may include a distal portion 108a and a proximal portion 108b. Distal portion 108a of neuromodulation catheter 102 includes a plurality of expandable members 110 (“expandable members 110”).

Although distal portion 108a is shown in FIG. 1 as including two expandable members 110, in other examples, distal portion 108a may include three or more expandable members 110. Generally, distal portion 108a includes a plurality of expandable members 110.

Elongated member 108 may have any suitable outer diameter, and the diameter can be constant along the length of elongated member 108 or may vary along the length of elongated member 108. In some examples, elongated member 108 can be 2, 3, 4, 5, 6, or 7 French or another suitable size.

Distal portion 108a of elongated member 108 is configured to be advanced within an anatomical lumen of a human patient to locate expandable members 110 at a target treatment site within or otherwise proximate to the anatomical lumen. For example, elongated member 108 may be configured to position expandable members 110 within a blood vessel, a ureter, a duct, an airway, or another naturally occurring lumen within the human body. The examples described herein focus on the anatomical lumen being a blood vessel, such as a renal vessel, but it will be understood that similar techniques may be used with other anatomical lumens. In certain examples, intravascular delivery of the expandable members 110 includes percutaneously inserting a guidewire (not shown in FIG. 1) into a vessel of a patient and moving elongated member 108 and/or expandable members 110 along the guidewire until expandable members 110 reach a target treatment site (e.g., a renal artery). For example, distal end 108a of elongated member 108 may define a passageway for engaging the guidewire for delivery of expandable members 110 using over-the-wire (OTW) or rapid exchange (RX) techniques. In other examples, neuromodulation catheter 102 can be a steerable or non-steerable device configured for use without a guidewire. In still other examples, neuromodulation catheter 102 can be configured for delivery via a guide catheter or sheath (not shown in FIG. 1), or other guide device.

Once at the target treatment site, expandable members 110 can be configured to deliver therapy, such as RF energy or cryogenic cooling, to provide or facilitate neuromodulation therapy at the target treatment site. For ease of description, the following discussion will be primarily focused on delivering RF energy, in which example expandable members 110 include one or more electrodes. It will be understood, however, that expandable members 110 may include elements or structures configured to deliver other types of therapy. For example, expandable members 110 may include ultrasound transducers configured to deliver ultrasound energy for ultrasound ablation of nerves near the vessel in which neuromodulation catheter 102 is positioned. As another example, expandable members 110 may include one or more slots defined in expandable members 110 and an expandable structure, such as a balloon, within a lumen of expandable members 110. The expandable structure, such as the balloon, may be filled with a cryogenic fluid to cause cryogenic cooling of tissue adjacent to the one or more slots.

Each expandable member 110 may include a corresponding one or more electrodes (not shown in FIG. 1) arranged along the length of the expandable member 110. For instance, each expandable member 110 may include two or three electrodes arranged along the length of the expandable member 110.

Each expandable member 110 may be configured to assume a delivery configuration in which expandable member 110 defines a relatively smaller radial extent, and a radially expanded configuration in which expandable member 110 defines a relatively larger radial extent. Distal portion 108a may be delivered through vasculature of the patient to the target treatment site while in the delivery configuration. In the radially expanded helical configuration, each expandable member 110 is configured to position the corresponding one or more therapeutic elements, such as electrodes or cryogenic elements, carried by the respective therapeutic element near a vessel wall, e.g., in apposition to the vessel wall. The therapeutic elements may be positioned along each expandable member 110 such that, when expandable members 110 are in the radially expanded configuration, the therapeutic elements are spaced around an inner perimeter (e.g., circumference) of the vessel wall. In some examples, the therapeutic elements may be positioned along each expandable member 110 such that, when expandable members 110 are in the radially expanded configuration, the therapeutic elements are substantially evenly spaced around an inner perimeter (e.g., circumference) of the vessel wall.

The therapeutic elements also may be positioned along each expandable member 110 such that, when expandable members 110 are in the radially expanded configuration, the therapeutic elements are located at a substantially similar longitudinal position within the vessel. For instance, the therapeutic elements also may be positioned along each expandable member 110 such that, when expandable members 110 are in the radially expanded configuration, at least part of each electrode being is intersected by a plane substantially orthogonal to a longitudinal axis of the elongated member. In this way, neuromodulation catheter 102 may enable formation of substantially circumferentially continuous lesions around the vessel wall.

In some implementations, distal portions of expandable members 110 may be connected (e.g., fused, adhered, attached via a mechanical attachment mechanism, such as a ring, formed as a unitary body, or the like) at a distal tip of neuromodulation catheter 100. As this results in a single distal tip for neuromodulation catheter 100, this may facilitate introduction of neuromodulation catheter 100 through vasculature of the patient, e.g., in examples in which neuromodulation catheter 100 is not introduced within a guide sheath. Further, in some implementations, having distal portions of expandable members 110 may be connected at a distal tip of neuromodulation catheter 100 may reduce an extension of expandable members 110 when expandable members 110 are in the radially expanded configuration. In examples in which distal portions of expandable members 110 are connected to each other, the expandable members 110 may define a common (e.g., single) expandable body with two (or more) expandable portions.

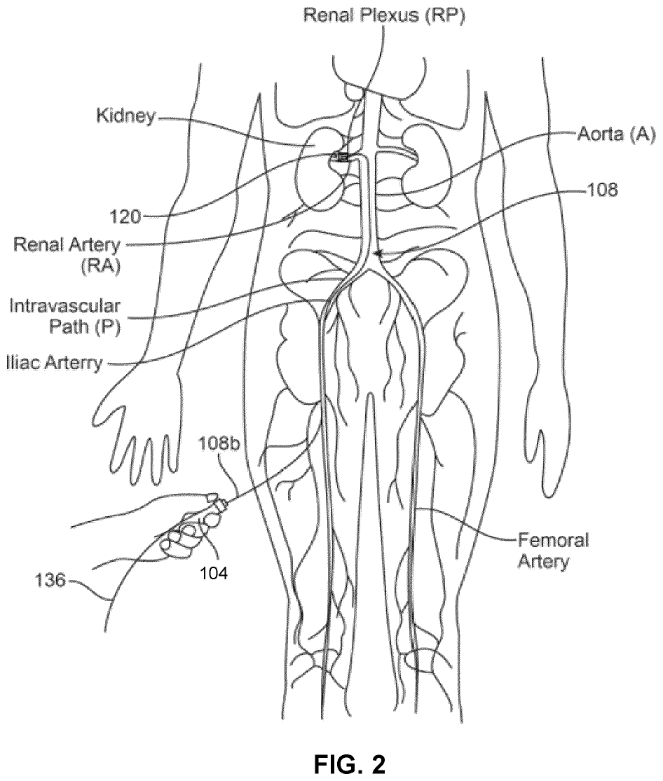

FIG. 2 (with additional reference to FIG. 1) illustrates an example technique for gaining access to renal nerves of an example patient in accordance with some examples of the present technology. FIG. 2 and following will be described with primary reference to RF neuromodulation and electrodes as the therapeutic elements. However, it will be appreciated that similar devices, systems, and techniques may be adapted for neuromodulation using other modalities, such as ultrasound, cryotherapy, direct heating, or the like. Neuromodulation catheter 102 provides access to the renal plexus (RP) through an intravascular path (P), such as a percutaneous access site in the femoral (illustrated), brachial, radial, or axillary artery to a target treatment site within a respective renal artery (RA). By manipulating proximal portion 108b of elongated member 108 from outside the intravascular path (P), a clinician may advance at least distal portion 108a of elongated member 108 through the sometimes-tortuous intravascular path (P) and remotely manipulate distal portion 108a (FIG. 1) of elongated member 108. Distal portion 108a may be remotely manipulated by the clinician using the handle 104.

In the example illustrated in FIG. 2, expandable members 110 are delivered intravascularly to the treatment site using a guidewire 136 in an OTW technique. As noted previously, the neuromodulation assembly 120 may define a passageway for receiving guidewire 136 for delivery of the neuromodulation catheter using either an OTW or a RX technique. At the treatment site, guidewire 136 can be at least partially withdrawn or removed, and expandable members 110 can transform or otherwise be moved to a deployed arrangement (e.g., the radially expanded helical configuration) for delivering RF energy. In other examples, expandable members 110 may be delivered to the treatment site within a different guide device, such as guide sheath (not shown in FIG. 2), with or without using guidewire 136. In examples in which the system includes a guide sheath, when expandable members 110 are at the target treatment site, the guide sheath may be at least partially withdrawn or retracted and expandable members 110 may be transformed into the deployed arrangement. For example, at least a portion of expandable members 110 may be self-expandable such that they expand to the deployed arrangement upon being released from the guide sheath. In still other examples, elongated member 108 may be steerable itself such that expandable members 110 may be delivered to the treatment site without the aid of guidewire 136 and/or a guide sheath.

Renal modulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for a period of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end state renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced, thermally induced, chemically induced, or induced in another suitable manner or combination of manners at one or more suitable target treatment sites during a treatment procedure. The target treatment site can be within or otherwise proximate to a renal lumen (e.g., a renal artery, a ureter, a renal pelvis, a major renal calyx, a minor renal calyx, or another suitable structure), and the treatment tissue can include tissue at least proximate to a wall of the renal lumen. For example, with regard to a renal artery, a treatment procedure can include modulating nerves in the renal plexus, which lay intimately within or adjacent to the adventitia of the renal artery. The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of treatment devices and associated methods for achieving renal neuromodulation via intravascular access and impose specific design requirements for such devices. Specific design requirements may include accessing the renal artery, positioning expandable members 110 within the renal artery, delivering the therapy to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

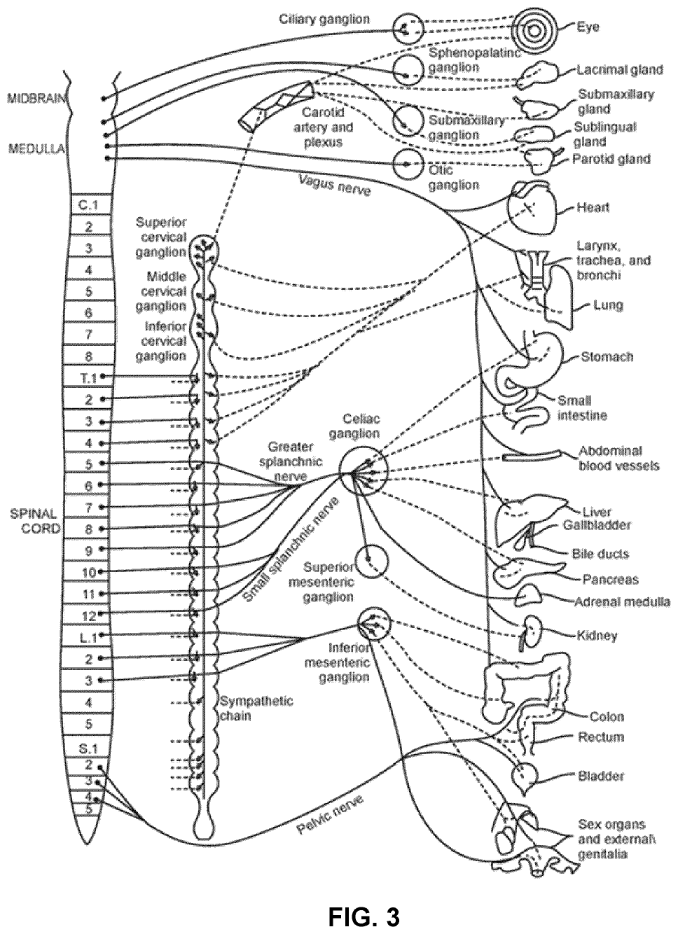

As shown in FIG. 3, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are through to extend to the second or third lumbar segments. Because SNS cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they separate out through white rami connectors which connect to either the paravertebral (which lie near the vertebral column) or prevertebral (which lie near the aortic bifurcation) ganglia extending alongside the spinal column.

To reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other component of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cell of the SNS is located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle, and inferior), which send sympathetic nerve fibers to the head and thorax organs, and the celiac and mesenteric ganglia (which send sympathetic fibers to the gut).

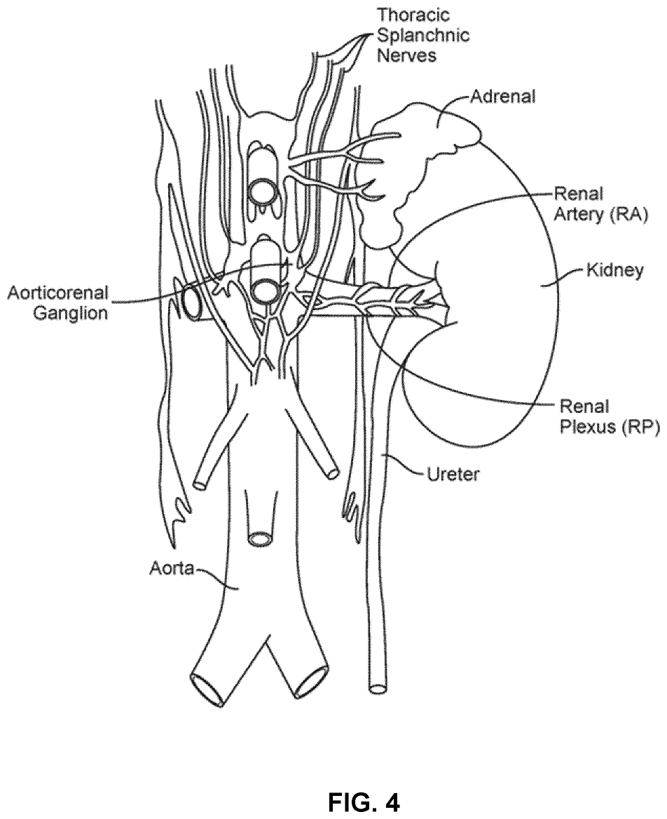

As FIG. 4 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery and is embedded within the adventitia of the renal artery. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); or raise blood pressure. Afferent messages carry signals from various organs and sensory receptors in the body to other organs and, particularly, the brain.

Hypertension, heart failure, and chronic kidney disease are a few of the many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure) and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end state renal disease in some patients are characterized by heightened sympathetic nervous activation. In patients with end state renal disease, plasma levels of norepinephrine above the median have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus and the renal tubules. Stimulation of the renal sympathetic nerves causes increased renin release, increased sodium (Na+) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects, and others.

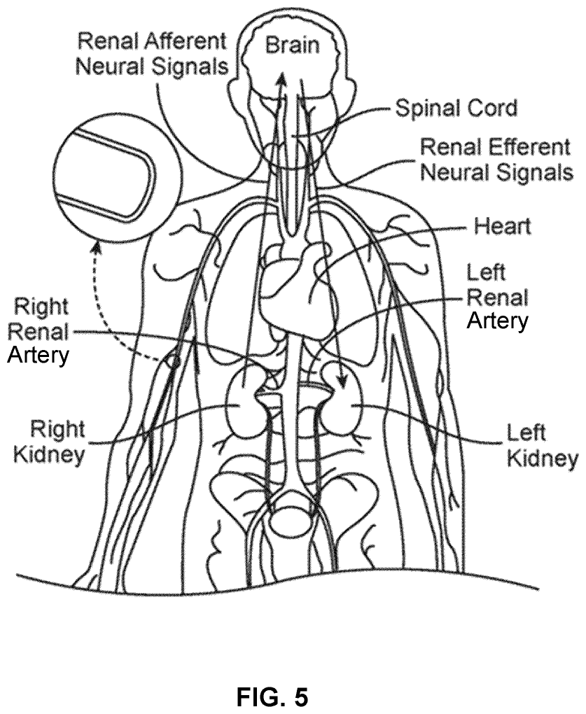

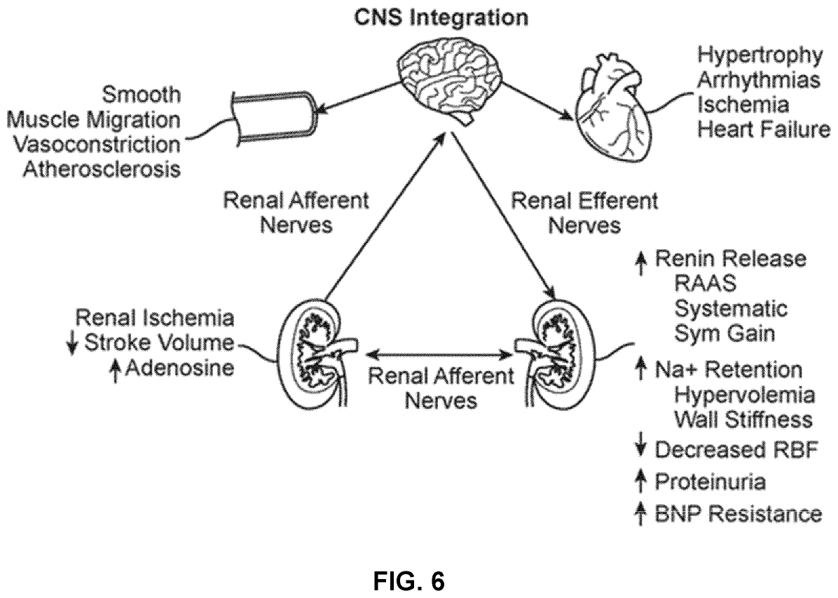

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 5 and 6, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end state renal disease, inappropriate fluid retention in heart failure, cardio-renal syndrome, and sudden death. Since the reduction of afferent neural signals contributing to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 5. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

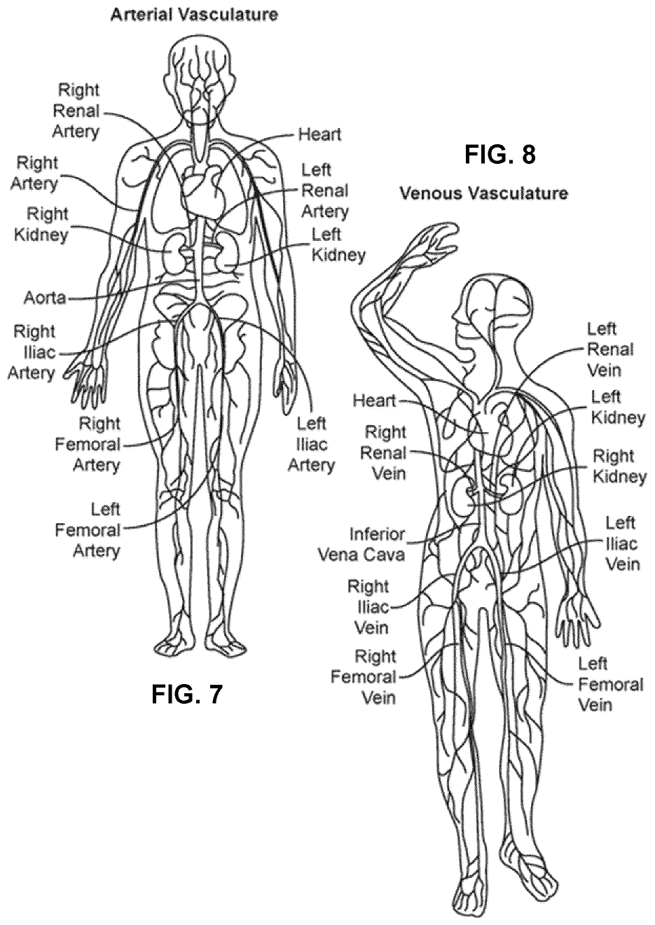

In accordance with the present technology, neuromodulation of a left and/or right renal plexus (RP), which is intimately associated with a left and/or right renal artery, may be achieved through intravascular access. As FIG. 7 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 8 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic techniques. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

The neuromodulatory apparatus may also be configured to allow for adjustable positioning and repositioning of the expandable members 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that expandable members 110 may intimately contact the vessel wall and/or extend at least partially through the vessel wall. Renal artery vessel diameter, DRA, typically is in a range of about 2-10 millimeters (mm), with most of the patient population having a DRA of about 4 mm to about 8 mm and an average of about 6 mm. Renal artery vessel length, LRA, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the artery) to avoid non-target tissue and anatomical structures such as anatomical structures of the digestive system of psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and aorta may vary significantly between patients, and also may vary dynamically within a patient, e.g., due to kidney motion. The take-off angle generally may be in a range of about 30°-135°.

In accordance with examples of the current disclosure, a neuromodulation catheter may include expandable members configured to position energy delivery elements, such as electrodes, around an inner perimeter of a blood vessel. The energy delivery elements also may be positioned along each expandable member such that, when the expandable members are in the radially expanded helical configuration, the electrodes are located at a substantially similar longitudinal position within the vessel. In this way, the neuromodulation catheter may facilitate formation of circumferential lesions around a blood vessel, such as a continuous circumferential lesions around a blood vessel.

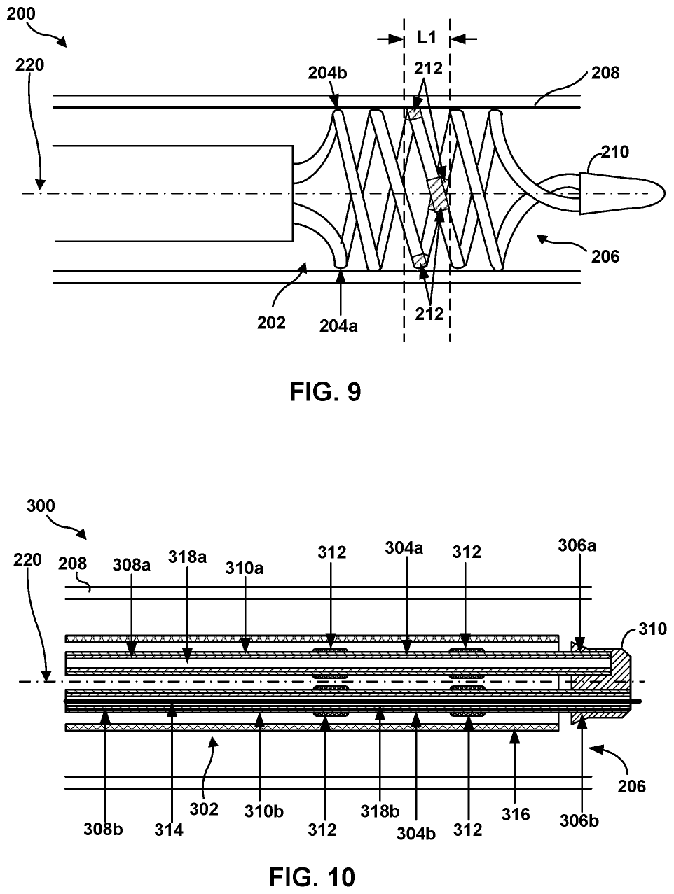

FIG. 9 is a conceptual diagram of an example neuromodulation catheter 200 that includes an elongated member. Neuromodulation catheter 200 is an example of neuromodulation catheter 102 of FIG. 1. Neuromodulation catheter 200 also includes a plurality of expandable members 204a and 204b (collectively, “expandable members 204” or individually “expandable member 204”) positioned at distal portion 202. FIG. 9 illustrates a distal portion 202 of the elongated member positioned within a renal vessel 206. Renal vessel 206 includes vessel wall 208. In some instances, distal portion 202 may be positioned in a main renal artery, an accessory renal artery, or a branch vessel extending distally from a main renal artery or accessory renal artery. In other examples, distal portion 202 may be positioned within a different blood vessel, such as a renal vein or another non-renal vessel.

Neuromodulation catheter 200 is configured to deliver a therapy via expandable members 204. For example, neuromodulation catheter 200 may be configured to deliver RF energy via the plurality of expandable members 204. As another example, neuromodulation catheter 200 may be configured to deliver ultrasound therapy or microwave therapy via the plurality of expandable members 204. The energy may be used to neuromodulate nerve tissue of the renal plexus adjacent to renal vessel 206 (e.g., by ablating the nerve tissue and creating lesions).

In the example shown in FIG. 9, neuromodulation catheter 200 includes two expandable members 204a and 204b. In other examples, neuromodulation catheter 200 may include more than two expandable members 204. Generally, neuromodulation catheter 200 may include a plurality of expandable members 204. Each expandable member 204 may have any suitable outer diameter and any suitable inner diameter, and the diameter(s) can be constant along the length of the expandable member 204 or may vary along the length of the expandable member 204.

Each expandable member 204 may be transformable between a delivery configuration and a radially expanded helical configuration. In the delivery configuration, each expandable member 204 may define a smaller radial extent. In the radially expanded helical configuration, each expandable member 204 defines a helical structure including a plurality of turns. The radially expanded helical configuration is shown in FIG. 9, while FIG. 10 illustrates an example of a neuromodulation catheter in a delivery configuration.

As shown in FIG. 9, in the radially expanded helical configuration, each expandable member 204 may define a helical structure with a helix axis parallel to a longitudinal axis 220 of neuromodulation catheter 200 (e.g., the helix axis may be coincident with longitudinal axis). For example, expandable member 204 may be configured such that, in the radially expanded helical configuration, the turns of the helix revolve around longitudinal axis 220 as expandable member 204 extends along the longitudinal axis 220. In some examples, expandable member s 204 may revolve in opposite directions from each other (e.g., first expandable member 204a may revolve in a clockwise direction while a second expandable member 204b may revolve in a counterclockwise direction, or vice versa). In the other examples, as shown in FIG. 9, expandable members 204 may rotate in the same direction.

Each expandable member 204 may trace any suitable number of helical revolutions in the radially expanded helical configuration. In the examples shown in FIG. 9, each expandable member 204 may trace two complete helical revolutions around longitudinal axis 220. In other examples, each expandable member 204 may trace one helical revolution around longitudinal axis 220, three or more helical revolutions around longitudinal axis 220, or a non-integer number of helical revolutions. Each expandable member 204 may trace the same number of helical revolutions, or one or more expandable members 204 may trace a different number of helical revolutions around the longitudinal axis 220 than one or more other expandable members 204.

Expandable members 204 may be configured to accommodate a variety of vessel diameters. For example, renal vessels may have a diameter between about 3 mm and about 8 mm. Other vessels may have other diameters. Because expandable members 204 assume a radially expanded helical configuration in a deployed state, expandable members 204 may accommodate different vessel diameters by assuming helices with different helical diameters. In this way, a single neuromodulation catheter 200 may be used to deliver therapy to vessels with different diameters, e.g., diameters in a range of between about 3 mm and about 8 mm.

In some examples, each expandable member 204 may be a separate component. For example, each expandable member 204 may include a separate shaft including, for example, a shape memory structure, an outer jacket surrounding the shape memory structure, and, optionally, an inner liner. In examples in which each expandable member 204 is a separate component, a proximal portion of each of the plurality of expandable members 204 may be joined to the elongated member and a distal portion of each of the plurality of expandable members 204 may be connected at a distal tip 210 of neuromodulation catheter 200.

In other examples, expandable members 204 may be a single component. For instance, the expandable members 204 may include a single shape memory component that is unitary at a proximal and/or distal portion of the single shape memory component and splits into a number of generally longitudinally extending portions, each generally longitudinally extending portion corresponding to an expandable member of expandable members 204. The single shape memory component may be shaped (e.g., heat set) into a shape with a number of helices corresponding to the number of expandable members 204. Each generally longitudinally extending portion may be covered with an outer jacket, and may optionally have an inner liner disposed within a lumen of the generally longitudinally extending portion.

Each expandable member 204 includes two or more corresponding electrodes 212. In the example shown in FIG. 9, each expandable members 204 include two electrodes 212. In other examples, each expandable member 204 made include three or more electrodes 212. expandable members 204 may include the same number of electrodes 212 or may include a different number of electrodes 212. In some examples, two or more of electrodes 212 of a particular expandable member 204 or different expandable members are configured to be separately electrically connected to an energy source, such that control circuitry of a therapy delivery device can independently control energy deliver to each of the electrodes. For example, all of the electrodes 212 of catheter 200 can be configured to be independently controllable. in other examples, two or more electrodes 212 are electrically coupled and are configured to be controlled by the control circuitry as a group.

The two or more electrodes 212 are positioned along the length of the corresponding expandable member 204. The two or more electrodes 212 may be positioned along each expandable member 204 such that, when expandable members 204 are in the radially expanded helical configuration, the electrodes are spaced around an inner perimeter (e.g., circumference) of vessel wall 208. In some examples, the two or more electrodes 212 may be positioned along each expandable member 204 such that, when expandable members 204 are in the radially expanded helical configuration, the two or more electrodes 212 are substantially evenly spaced around an inner perimeter (e.g., circumference) of vessel wall 208. For instance, in examples in which each expandable member 204 includes two electrodes 212, the two or more electrodes 212 may be positioned along each expandable member 204 such that, when expandable members 204 are in the radially expanded helical configuration, the electrodes 212 of a single expandable member 204 are spaced approximately 180 degrees apart from each other around the inner perimeter of vessel wall 208. Further, the two or more electrodes 212 may be positioned along each expandable member 204 relative to each other such that, when expandable members 204 are in the radially expanded helical configuration, all the electrodes 212 of the two expandable members 204 are spaced approximately 90 degrees apart from each other around the inner perimeter of vessel wall 208.

The electrodes 212 also may be positioned along each expandable member 204 such that, when expandable members 204 are in the radially expanded helical configuration, the electrodes 212 are located at a substantially similar longitudinal position within renal vessel 206. The longitudinal positioning of electrodes 212 may be characterized using a deployed electrode length. As used herein, the deployed electrode length is a distance between, in the radially expanded helical configuration, a proximal-most point of a proximal-most electrode of the plurality of electrodes 212 and a distal-most point of a distal-most electrode of the plurality of electrodes 212.

A smaller deployed electrode length indicates a more longitudinally compressed deployment of electrodes 212, and, thus, a more circular arrangement of electrodes when expandable members 204 of neuromodulation catheter 200 are in the radially expanded helical configuration. Conversely, a larger deployed electrode length indicates a less longitudinally compressed deployment of electrodes 212, and, thus, a more elongated helical arrangement of electrodes 212 when expandable members 204 are in the radially expanded helical configuration. In FIG. 9, the deployed electrode length is labelled L1. In some examples, neuromodulation catheter 200 defines a deployed electrode length, L1, of between about 1 mm and about 10 mm, or about 1 mm and about 7 mm, or between about 1 mm and about 5 mm, or between about 1 mm and about 3 mm.

In this way, neuromodulation catheter 200 may enable formation of substantially circumferentially continuous lesions around vessel wall 208. For instance, electrodes 212 may be positioned along each expandable member 204 such that, when expandable members 204 are in the radially expanded helical configuration, at least part of each electrode being is intersected by a plane substantially orthogonal to a longitudinal axis of the elongated member.

In some examples, electrode 212 is manufactured separately from other components of catheter 200 and incorporated into neuromodulation catheter 200 after manufacture. In other examples, electrode 212 is formed as part of another component of catheter 200. For example, in some examples, electrode 212 is formed by incorporating electrically conductive (e.g., metal) elements into expandable member 204 (e.g., in a polymer portion of expandable member 204). In other examples, electrode 212 may be formed by incorporating gold (or another electrically conductive material) into expandable member 204 (e.g., through a gold electroplating technique or other suitable deposition technique).

As described above, distal portions of expandable members 204 are connected at distal tip 210. Distal tip 210 may be configured to join expandable members 204 to present a single tip at the distal-most portion of neuromodulation catheter 200. Further, by constraining movement of the distal portions of expandable members 204 relative to each other, distal tip 210 may help control longitudinal extension and contraction of expandable members 204 when expandable members 204 transition from the delivery configuration to the radially expanded helical configuration, and vice versa. Distal tip 210 may be made of a biocompatible polymer. The polymer may include, for example, a thermoplastic, such as an elastomer. In some examples, the elastomer may include a polyurethane, a silicone, or a copolymer, such as a block including polyamide and polyether available under the trade name Pebax® available from Arkema S.A., Colombes, France. In some examples, distal tip 210 may be formed from a polymer with a relatively low Shore hardness so distal tip 210 presents a relatively atraumatic tip in case of contact between distal tip 210 and tissue.

In some examples, distal tip 210 may have a relatively extended longitudinal length. This may facilitate advancing of neuromodulation catheter 200 through vasculature of a patient, e.g., in the absence of a guidewire. In some examples, the length of distal tip 210, measured parallel to longitudinal axis 220, may be between about 5 mm and about 2 cm.

As described above, FIG. 9 illustrates neuromodulation catheter 200 with expandable members in a radially expanded helical configuration. FIG. 10 illustrates a schematic cross-sectional view of an example neuromodulation catheter 300 with expandable members 304 in a delivery configuration. Neuromodulation catheter 300 may be similar to or substantially the same as neuromodulation catheter 200 of FIG. 9, aside from the differences described herein.

Distal portion 302 of neuromodulation catheter 300 includes two expandable members 304a, 304b (collectively, “expandable members 304” or individually “expandable member 304”), each of which includes two electrodes 312. Each expandable member 304a, 304b includes a corresponding distal portion 306a, 306b. Distal portions 306a, 306b are connected by distal tip 310.

As seen in FIG. 10, each expandable member 304 includes a corresponding shape memory structure 308a, 308b (collectively, “shape memory structures 308” or individually, “shape memory structure 308”) and a corresponding outer jacket 310a, 310b (collectively, “outer jackets 310” or individually, “outer jacket 310”). Each shape memory structure 308 includes a hollow tubular structure. In some examples, each shape memory structure 308 may be formed from a stranded or coiled wire formed into the hollow tubular structure, such as a helical hollow strand (HHS®) tube available from Fort Wayne Metals, Fort Wayne, Indiana.

In other examples, one or both shape memory structures 308 may have another configuration. For example, one or both shape memory structures 308 may include solid structures, such as a solid round wire, a solid flat wire, a solid elliptical wire, or the like. As another example, one or both shape memory structures 308 may include a stranded structure that does not define an inner lumen, such as a stranded wire. While the lumen defined by the hollow tubular structures shown in FIG. 10 may provide space for a guidewire, removing the lumen from one or both expandable members 304 may reduce a radial profile of the one or both expandable members 304.

Shape memory structures 308 may be shape set into a helical shape, such that when shape memory structures 308 are unconstrained by a guidewire 314 and/or guide sheath 316, shape memory structures 308 urge expandable members 304 to the radially expanded helical configuration.

Outer jackets 310 may circumferentially surround shape memory structures 308. Outer jackets 310 may include a biocompatible polymer. The polymer may include, for example, a thermoplastic, such as an elastomer. In some examples, the elastomer may include a polyurethane, a silicone, or a copolymer, such as a block including polyamide and polyether available under the trade name Pebax® available from Arkema S.A., Colombes, France.

As shown in FIG. 10, one or both shape memory structures 308 may define an internal lumen 318a, 318b (collectively, “internal lumens 318” or individually, “internal lumen 318”). Internal lumens 318 may define a void volume within expandable members 304 through which a guidewire 314 may be advance, through which electrical connections may travel, or the like. In the example shown in FIG. 10, one guidewire 314 is within internal lumen 318b of one of expandable members 304.

Guidewire 314 may be configured to be advanced through vasculature of the patient to the target treatment site, and neuromodulation catheter 300 may be advanced over guidewire 314. Additionally, or alternatively, guidewire 314 may be configured to urge second expandable member 304b toward the delivery configuration, in which second expandable member 304b defines a smaller radial extent and may be substantially straight, as shown in FIG. 10. Because first expandable member 304a is connected to second expandable member 304b at distal tip 310 and at proximal ends of first and second expandable members 304a, 304b (e.g., connected by being connected to the elongated member), urging second expandable member 304b toward the delivery configuration also urges first expandable member 304a toward the delivery configuration. Although the example of FIG. 10 includes a single guidewire 314, in other examples, a corresponding guidewire 314 may extend through each internal lumen 318a, 318b. In other examples, the system that includes neuromodulation catheter 300 may omit guidewire 314.

In the example shown in FIG. 10, in the delivery configuration, first and second expandable members 304a, 304b are substantially parallel. In other examples, in the delivery configuration, first expandable member 304a (through which guidewire 314 does not extend) may wrap around second expandable member 304b (through which guidewire 314 extends).

FIG. 10 also shows distal portion 302 of neuromodulation catheter 300 within a guide sheath 316. In some examples, guide sheath 316 may be advanced through vasculature of a patient to the target treatment site and neuromodulation catheter 300 may be advanced through guide sheath 316 to the target treatment site. Alternatively, neuromodulation catheter 300 may be positioned within guide sheath 316, and neuromodulation catheter 300 and guide sheath 316 may be advanced vasculature of the patient to the target treatment site together. Guide sheath 316 may be used with or without guidewire 314.

In some examples, guide sheath 316 is configured to urge expandable members 304 toward the delivery configuration, e.g., by radially constraining expandable members 304. To cause the expandable members 304 to assume the radially expanded helical configuration, a clinician may cause relative movement between neuromodulation catheter 300 and guide sheath 316. For instance, while holding distal portion 302 of neuromodulation catheter 300 substantially stationary relative to vessel wall 208, the clinician may withdraw guide sheath 316 proximally to cause distal portion 302 to extend out a distal port of guide sheath 316. Alternatively, while holding guide sheath 316 substantially stationary relative to vessel wall 208, the clinician may advance distal portion 302 of neuromodulation catheter 300 distally to cause distal portion 302 to extend out a distal port of guide sheath 316. As such, in some examples, guide sheath 316 may be used without guidewire 314 or guidewire 314 may be used without guide sheath 316.

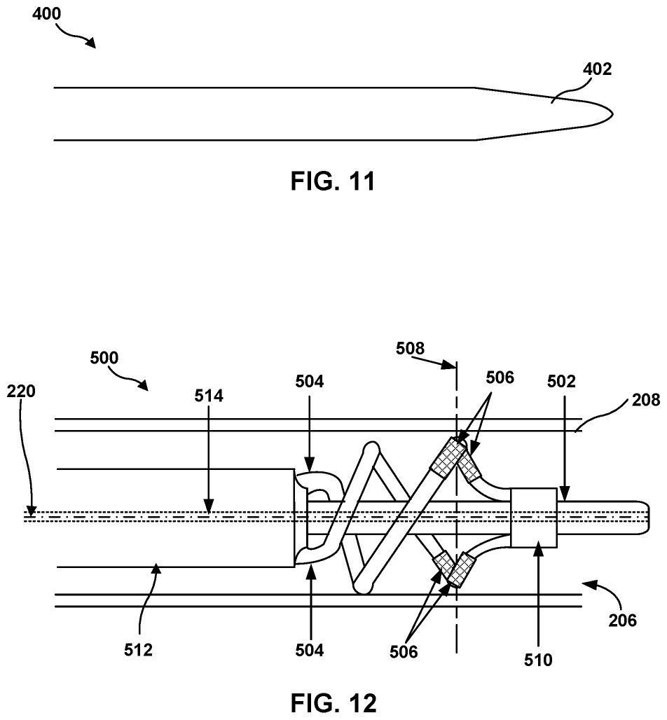

FIG. 11 is a conceptual diagram of an example guide sheath 400 in which a neuromodulation catheter may be positioned. As shown in FIG. 11, guide sheath 400 may include a longitudinally extended tapered tip 402. Longitudinally extended tapered tip 402 may facilitate advancing and steering of guide sheath 400 through vasculature of a patient and may allow use of guide sheath 400 without a guidewire. In some examples, a length of longitudinally extended tapered tip 402, measured parallel to a longitudinal axis of guide sheath 400 from a point at which guide sheath 400 begins to taper, may be between about 5 mm and about 2 cm.

In some examples, rather than a guidewire extending through one of expandable members, as shown in FIG. 10, a neuromodulation catheter may include a separate shaft and lumen through which a guidewire extends. FIG. 12 is a conceptual diagram of an example neuromodulation catheter 500 that includes a center guide member 502 through which a lumen 514 extends. Neuromodulation catheter 500 may be similar to or substantially the same as neuromodulation catheters 100, 200, and 300 of FIGS. 1, 9, and 10 aside from differences described herein. Like neuromodulation catheters 100, 200, and 300 of FIGS. 1, 9, and 10, neuromodulation catheter 500 includes a plurality of expandable members 504 (e.g., two expandable members 504), each of which includes two or more electrodes 506 (e.g., each of which includes two electrodes 506). In the example of FIG. 12, electrodes 506 are positioned along each of expandable members 504 such that, when expandable members 504 are in the radially expanded deployed shape, electrodes 506 are longitudinally positioned so that a single plane 508 substantially orthogonal to longitudinal axis 220 intersects each electrode 506. Also like neuromodulation catheters 100, 200, and 300 of FIGS. 1, 9, and 10, distal portions of expandable members 504 are connected at distal tip 510, and proximal portions of expandable members 504 are connected to the elongated member of neuromodulation catheter 500.

Unlike neuromodulation catheters 100, 200, and 300 of FIGS. 1, 9, and 10, neuromodulation catheter 500 includes center guide member 502. Center guide member 502 may extend from distal portion 512 of the elongate shaft of neuromodulation catheter 500 parallel to longitudinal axis 220. In some examples, center guide member 502 extends within a catheter lumen (not pictured) located within the elongate shaft of neuromodulation catheter 500. A clinician may manipulate an actuator at a proximal end of neuromodulation catheter 500 to extend or retract center guide member 502 relative to the distal portion 506.

As illustrated in FIG. 12, a distal tip 508 may be located on (e.g., attached to) the center guide member 502. In some such examples, by advancing or retracting center guide member 502 relative to distal portion 512 of the elongate shaft of neuromodulation catheter 500, a clinician may control the configuration of expandable members 504. For instance, by advancing center guide member 502 relative to distal portion 512, expandable members 504 may assume the delivery configuration, in which expandable members 504 wrap around center guide member 502 in a relatively low profile configuration. Conversely, by retracting center guide member 502 relative to distal portion 512, expandable members 504 may assume the radially expanded helical configuration, as shown in FIG. 12. In this way, by controlling a position of center guide member 502, a clinician may control a deployment state and a radial extent of therapeutic members 504.

In other examples, distal tip 510 may be independent from the center guide member 502 and form an annular structure around center guide member 502. By advancing or retracting distal tip 510 relative to distal portion 512 of the elongated member, a clinician may control the configuration of expandable members 504. For instance, by advancing distal tip 510 relative to distal portion 512, expandable members 504 may assume the delivery configuration, in which expandable members 504 wrap around center guide member 502 in a relatively low profile configuration. Conversely, by retracting distal tip 510 relative to distal portion 512, expandable members 504 may assume the radially expanded helical configuration, as shown in FIG. 12. In this way, by controlling a position of distal tip 510, a clinician may control a deployment state and radial extent of therapeutic members 504.

In some examples, center guide member 502 is radially centered relative to the elongate shaft of neuromodulation catheter 500 and/or relative to distal tip 508. In other examples, center guide member 502 is radially offset from the center longitudinal axes of the elongate shaft and/or distal tip 508.

As shown in FIG. 12, in some examples, distal portion 512 of neuromodulation catheter 500 and center guide member 502 define a lumen 514, which may be radially centered in catheter 500 and/or guide member 502, or may be radially offset from a central longitudinal axis of catheter 500 and/or guide member 502. In some examples, lumen 514 is configured to receive a guidewire (not pictured). Thus, in some examples, lumen 514 enables a guidewire to be positioned within neuromodulation catheter 500, e.g., to facilitate navigation of neuromodulation catheter 500 to a target treatment site over the guidewire. By including center guide member 502 defining lumen 514, the guidewire may not need to pass through the helically shaped configuration of expandable members 504.

In other examples, instead of or in addition to defining a guidewire lumen 514, center guide member 502 is configured to function as a positioning element (or also referred to as a guide element) configured to facilitate navigation of neuromodulation catheter 500 to a target treatment site without the use of a guidewire. For example, center guide member 502 may be configured to extend distally past a distalmost end of distal tip 508, such that it defines a distal guide element having a sufficient stiffness to aid with navigation of catheter 500 through vasculature of a patient. In some examples, the distal guide element defined by a distal portion of guide member 502 is tapered or otherwise defines an atraumatic tip. In some of these examples, guide member 502 is formed from a solid member that does not define a lumen or a structure that does not otherwise define a distal opening. In other of these examples, however, guide member 502 defines an inner lumen, through which a fluid, such as a contrast agent, may be delivered to a target site within vasculature of a patient.

In some examples, rather than including expandable members that are connected at their distal portions, a neuromodulation catheter may include a plurality of expandable members configured to assume a radially expanded curved configuration, where, in the radially expanded curved configuration, each expandable member traces an arc of less than about 360 degrees. Like the other examples described herein, this may facilitate positioning of electrodes around a perimeter of a vessel with a relatively short deployed electrode length.

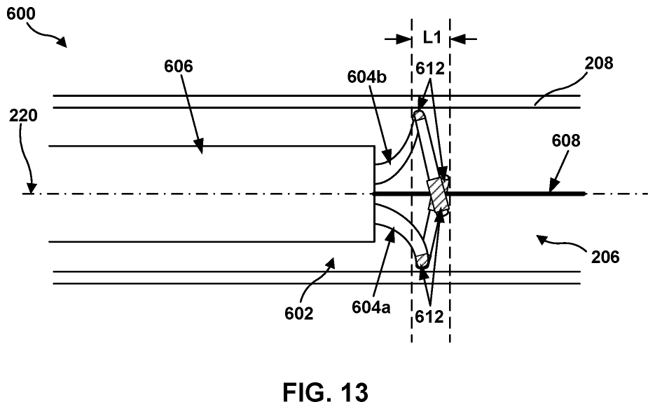

FIG. 13 is a conceptual diagram illustrating another example neuromodulation catheter 600, which includes a plurality of expandable members 604 (collectively, “expandable members 604” or individually “expandable member 604”) at distal portion 602 of neuromodulation catheter 600, in accordance with some examples of the present disclosure. As shown in FIG. 13, each expandable member of plurality of expandable members 604 may be configured to assume a radially expanded curved configuration when released from a guide sheath 606. FIG. 13 also illustrates a guidewire 608 over which neuromodulation catheter 600 may be advanced, and which extends through an internal lumen of neuromodulation catheter 600.

In the example shown in FIG. 13, neuromodulation catheter 600 includes two expandable members 604a and 604b. In other examples, neuromodulation catheter 600 may include more than two expandable members 604. Generally, neuromodulation catheter 600 may include a plurality of expandable members 604. Each expandable member 604 may have any suitable outer diameter and any suitable inner diameter, and the diameter(s) can be constant along the length of the expandable member 604 or may vary along the length of the expandable member 604.

Each expandable member 604 may be transformable between a delivery configuration and a radially expanded curved configuration. In the delivery configuration, each expandable member 604 may define a smaller radial extent. In the radially expanded curved configuration, each expandable member 604 defines a curved structure tracing an arc length.

In the radially expanded curved configuration, each expandable member 604 may define a curved structure with an axis parallel to a longitudinal axis 220 of neuromodulation catheter 600 (e.g., the curved structure axis may be coincident with longitudinal axis). In some examples, expandable members 604 may curve in opposite directions from each other (e.g., first expandable member 604a may curve in a clockwise direction while second expandable member 604b may curve in a counterclockwise direction, or vice versa). In the other examples, expandable members 604 may curve in the same direction.

Each expandable member 604 may trace any suitable arc length. For instance, each expandable member 604 may trace an arc with a length of less than 360 degrees (less than one full revolution. As other examples, each expandable member 604 may trace an arc with a length of less than 270 degrees, or about 180 degrees, as shown in FIG. 13. Each expandable member 604 may trace the same arc length, or one or more expandable members 604 may trace a different arc length than one or more other expandable members 604.

In some examples, each expandable member 604 may be a separate component. For example, each expandable member 604 may include a separate shaft including, for example, a shape memory structure, an outer jacket surrounding the shape memory structure, and, optionally, an inner liner. In examples in which each expandable member 604 is a separate component, a proximal portion of each of the plurality of expandable members 604 may be joined to the elongated member.

Each expandable member 604 includes one or more corresponding electrodes 612. In the example shown in FIG. 13, each expandable member 604 include two electrodes 612. In other examples, each expandable member 604 may include one electrode or three or more electrodes 612. In an implementation, each expandable member 604 includes four electrodes. expandable members 604 may include the same number of electrodes 612 or may include a different number of electrodes 612.

The two or more electrodes 612 are positioned along the length of the corresponding expandable member 604. The two or more electrodes 612 may be positioned along each expandable member 604 such that, when expandable members 604 are in the radially expanded curved configuration, the electrodes 612 are spaced around an inner perimeter (e.g., circumference) of vessel wall 208. In some examples, the two or more electrodes 612 may be positioned along each expandable member 604 such that, when expandable members 604 are in the radially expanded helical configuration, the one or more electrodes 612 are substantially evenly spaced around an inner perimeter (e.g., circumference) of vessel wall 208. For instance, in examples in which each expandable member 604 includes two electrodes 612, the two or more electrodes 612 may be positioned along each expandable member 604 such that, when expandable members 604 are in the radially expanded curved configuration, the electrodes 612 of a single expandable member 604 are spaced approximately 90 degrees apart from each other around the inner perimeter of vessel wall 208. Further, the one or more electrodes 612 may be positioned along each expandable member 604 relative to each other such that, when expandable members 604 are in the radially expanded curved configuration, all the electrodes 6of the two expandable members 604 are spaced approximately 90 degrees apart from each other around the inner perimeter of vessel wall 208.

In some examples, the electrodes 612 are positioned along each expandable member 604 such that, when expandable members 604 are in the radially expanded helical configuration, the electrodes 612 are located at a substantially similar longitudinal position within renal vessel 206. The longitudinal positioning of electrodes 612 may be characterized using a deployed electrode length. As described above, the deployed electrode length is a distance between, in the radially expanded helical configuration, a proximal-most point of a proximal-most electrode of the plurality of electrodes 612 and a distal-most point of a distal-most electrode of the plurality of electrodes 612.

In FIG. 13, the deployed electrode length is labelled L1. In some examples, neuromodulation catheter 600 defines a deployed electrode length, L1, of between about 1 mm and about 10 mm, or about 1 mm and about 7 mm, or between about 1 mm and about 5 mm, or between about 1 mm and about 3 mm.

In this way, neuromodulation catheter 600 may enable formation of substantially circumferentially continuous lesions around vessel wall 208. For instance, electrodes 612 may be positioned along each expandable member 604 such that, when expandable members 604 are in the radially expanded helical configuration, at least part of each electrode being is intersected by a plane substantially orthogonal to a longitudinal axis of the elongated member.

Expandable members 604 may be configured to be deployed using any suitable mechanism, including any mechanism described herein. For instance, expandable member s 604 may include shape memory structures configured to urge expandable members 604 toward the radially expanded curved configuration upon being released from guide sheath 606, upon a guidewire being withdrawn from an internal lumen of one or both of expandable members 604, or both. As another example, expandable members 604 may be coupled to a push wire or pull wire and actuation of the push wire or pull wire may transform expandable members 604 between the delivery configuration and radially expanded curved configuration.

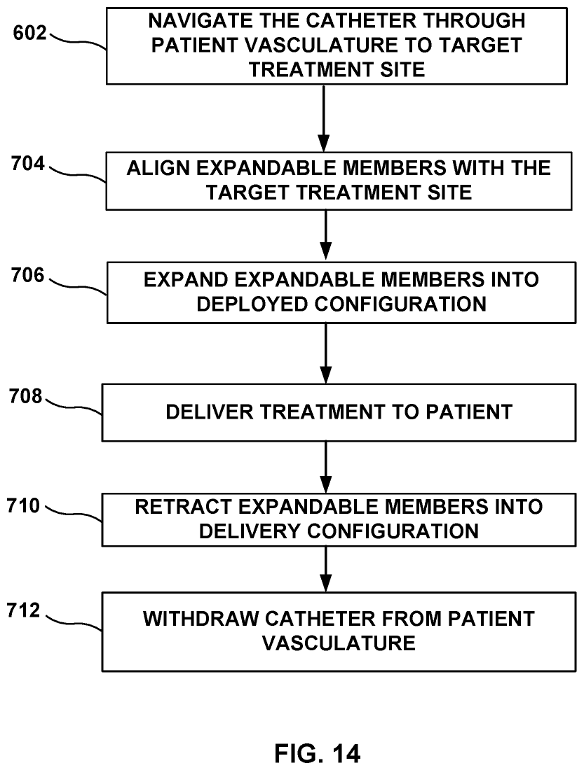

FIG. 14 illustrates an example technique for using the example neuromodulation catheters described herein to deliver a treatment to tissue of a patient while positioned within an anatomical lumen, such as renal vessel 206. The technique of FIG. 14 will be described with concurrent reference to neuromodulation catheter 200 of FIG. 9. However, a person having ordinary skill in the art will understand that the technique of FIG. 14 may be performed using any of the neuromodulation catheters described herein, and the neuromodulation catheters described herein may be used for other techniques.

A clinician first navigates neuromodulation catheter 200 through vasculature of the patient to a target treatment site (702). In some examples, the clinician may navigate neuromodulation catheter 200 to the target treatment site through a femoral artery approach, a brachial artery approach, a radial artery approach, or the like. The clinician may navigate the neuromodulation catheter 200 through patient vasculature using handle 104 of catheter 102 (FIG. 1), guidewire 314, guide sheath 316, or any other element or combination of elements configured to facilitate navigation of neuromodulation catheter 200 through the patient vasculature. The target treatment site may be any location at which the clinician intends to deliver a neuromodulation treatment such as, but is not limited to, a renal vessel and tissues near vessel wall 208 of the renal vessel. In some examples, a clinician may select a plurality of target treatment sites for treatment.

The clinician then aligns one or more expandable members 204 with the target treatment site (704). The clinician may align one or more expandable members 204 with the target treatment site such that when the expandable members 204 are transformed into the deployed configuration, one or more electrodes 212 contact vessel wall 208 at the target treatment site. In some examples, the clinician may align one or more expandable members 204 with the target treatment site after transforming the expandable members 204 into a partially deployed or completely deployed configuration.

The clinician then expands expandable members 204 into the radially expanded deployed configuration (e.g., the radially expanded helical configuration as shown in FIGS. 9, 10, and 12; or the radially expanded curved configuration as shown in FIG. 13) (706). expandable members 204 may be deployed using any of the examples described herein, such as removing a guidewire from a lumen of one or both expandable members 204 (FIG. 10), retracting a guide sheath relative to expandable members 204 (FIG. 10), extending expandable members 204 relative to a guide sheath (FIG. 10), or moving a distal tip relative to a distal portion of the neuromodulation catheter (FIG. 12).

Once the expandable members 204 are deployed, the clinician may deliver a treatment to the target treatment site through the electrodes 212 of the expandable members 204 (708). For example, the clinician may use an RF generator to deliver RF energy through the electrodes 202 to the tissue adjacent vessel wall 208. The RF energy may ablate tissue including renal nerves at the target treatment site. In other examples, instead of, or in addition to, RF energy, expandable members 204 may be configured to deliver one or more other types of neuromodulation energy to the target treatment, including, but not limited to, one or more of: microwave energy, ultrasound energy, optical energy, a chemical agent, radiation, cryogenic cooling, or the like.

After the treatment is delivered, the clinician may then retract expandable members 204 back into the delivery configuration (710), e.g., by performing the reverse operation used to extend expandable members 204 into the radially expanded helical configuration. The clinician then may withdraw neuromodulation catheter 200 from the patient vasculature (612).

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within a single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique or any combination thereof.

Moreover, unless the word “or” is expressly limited to mean only a single term exclusive from the other items in reference to a list of two or more items, then the use of “or” in such a list is to be interpreted as including (a) any single item in list, (b) all of the items in the list, or (c) any combination of the items in the list. Additionally, the terms “about” or approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Further disclosed herein is the subject-matter of the following clauses:

1. A system comprising:
a catheter comprising:
   an elongated member configured to be navigated through vasculature of a patient to a target treatment site; and
   a plurality of expandable members arranged at a distal portion of the elongated member, each expandable member comprising a corresponding two or more therapeutic elements arranged along a length of the expandable member, each expandable member of the plurality of expandable members being configured to assume a delivery configuration and a radially expanded helical configuration, each expandable member being configured to position the corresponding two or more therapeutic elements near a vessel wall so that the therapeutic elements are spaced around a perimeter of the vessel wall, and wherein distal portions of the plurality of expandable members are connected to each other at a distal tip of the catheter.

2. The system of clause 1, wherein, when the plurality of expandable members are in the radially expanded helical configuration, at least part of each therapeutic element is intersected by a plane substantially orthogonal to a longitudinal axis of the elongated member.

3. The system of clause 1 or 2, wherein each helical structure rotates about a corresponding helical axis, and wherein one or more helical axis is substantially parallel to a longitudinal axis of the elongated member.

4. The system of clause 3, wherein a first helical structure of the helical structures rotates in a different direction than a second helical structure of the helical structures.

5. The system of any one of clauses 1 to 4, wherein the distal tip comprises an extended tapered tip configured to facilitate navigation of the elongated member and the plurality of expandable members through the vasculature of the patient.

6. The system of any one of clauses 1 to 5, wherein the plurality of expandable members consists of two expandable members.

7. The system of any one of clauses 1 to 6, further comprising a guide sheath.

8. The system of clause 7, wherein the plurality of expandable members are configured assume the radially expanded helical configuration when the plurality of expandable members extend from a distal port of the guide sheath.

9. The system of clause 7 or 8, wherein the guide sheath comprises an extended tapered tip configured to facilitate navigation of the guide sheath through the vasculature of the patient.

10. The system of any one of clauses 1 to 9, further comprising a guide member.

11. The system of clause 10, wherein the guide member comprises a guidewire, wherein one or more expandable members of the plurality of expandable members comprises an inner lumen, and wherein the guidewire is configured to be positioned within the inner lumen when the plurality of expandable members is in the delivery configuration.

12. The system of clause 10 or 11, wherein the plurality of expandable members are configured to transition from the delivery configuration to the radially expanded helical configuration in response to the guide member being withdrawn proximally from the one or more expandable members.

13. The system of any one of clauses 1 to 12, wherein the plurality of expandable members comprise a shape memory structure.

14. The system of clause 13, wherein the plurality of expandable members comprise a common shape memory structure, and wherein the shape memory structure is configured to urge the plurality of expandable members from the delivery configuration to the radially expanded helical configuration.

15. The system of clause 13, wherein each expandable member of the plurality of expandable members comprises a corresponding shape memory structure, and wherein the corresponding shape memory structures are configured to urge the corresponding expandable members from the delivery configuration to the radially expanded helical configuration.

16. A method comprising:
navigating a catheter through vasculature of a patient to a target treatment site, wherein the catheter comprises:
   an elongated member; and
   a plurality of expandable members arranged at a distal portion of the elongated member, each expandable member comprising a corresponding two or more therapeutic elements arranged along a length of the expandable member, and distal portions of the plurality of expandable members being connected to each other at a distal tip of the catheter;
expanding each expandable member of the plurality of expandable members from a delivery configuration to radially expanded helical configuration, wherein, when the plurality of expandable members are in the radially expanded helical configuration, the corresponding two or more therapeutic elements of each expandable member are positioned near a vessel wall so that the therapeutic elements are spaced around a perimeter of the vessel wall; and
delivering treatment to the vessel wall using the two or more therapeutic elements.

17. The method of clause 16, wherein, when the plurality of expandable members are in the radially expanded helical configuration, at least part of each therapeutic element is intersected by a plane substantially orthogonal to a longitudinal axis of the elongated member.

18. The method of clause 16 or 17, wherein each helical structure rotates about a corresponding helical axis, and wherein one or more helical axis is substantially parallel to a longitudinal axis of the elongated member.

19. The method of clause 18, wherein a first helical structure of the helical structures rotates in a different direction than a second helical structure of the helical structures.

20. The method of any one of clauses 16 to 19, wherein the distal tip comprises an extended tapered tip configured to facilitate navigation of the elongated member and the plurality of expandable members through the vasculature of the patient.

21. The method of any one of clauses 16 to 20, wherein the plurality of expandable members consists of two expandable members.

22. The method of any one of clauses 16 to 21, wherein navigating the catheter through vasculature of the patient to the target treatment site comprises introducing the catheter through a guide sheath.

23. The method of clause 22, wherein expanding each expandable member of the plurality of expandable members comprises causing relative movement between the catheter and the guide sheath so that the plurality of expandable members extend from a distal port of the guide sheath.

24. The method of clause 22 or 23, wherein the guide sheath comprises an extended tapered tip configured to facilitate navigation of the guide sheath through the vasculature of the patient.

25. The method of any one of clauses 16 to 24, wherein navigating the catheter through vasculature of the patient to the target treatment site comprises advancing the catheter over a guide member.

26. The method of clause 25, wherein the guide member comprises a guidewire, wherein one or more expandable members of the plurality of expandable members comprises an inner lumen, and wherein advancing the catheter over the guide member comprises advancing the catheter over the guidewire while the guidewire is positioned within the inner lumen while the plurality of expandable members are in the delivery configuration.

27. The method of clause 25 or 26, wherein expanding each expandable member of the plurality of expandable members comprises withdrawing the guide member proximally from the one or more expandable members.

28. The method of any one of clauses 16 to 27, wherein the plurality of expandable members comprise a shape memory structure.

29. The method of clause 28, wherein the plurality of expandable members comprise a common shape memory structure, and wherein expanding each expandable member comprises urging the plurality of expandable members from the delivery configuration to the radially expanded helical configuration using the common shape memory structure.

30. The method of clause 13, wherein each expandable member of the plurality of expandable members comprises a corresponding shape memory structure, and wherein expanding each expandable member comprises urging the corresponding expandable members from the delivery configuration to the radially expanded helical configuration using the corresponding shape memory structures.

31. A system comprising:
a catheter comprising:
   an elongated member configured to be navigated through vasculature of a patient to a target treatment site; and
   a plurality of expandable members arranged at a distal portion of the elongated member, each expandable member comprising a corresponding one or more therapeutic elements arranged along a length of the expandable member, each expandable member of the plurality of expandable members being configured to assume a delivery configuration and a radially expanded curved configuration, each expandable member being configured to, in the radially expanded curved configuration, position the corresponding one or more therapeutic elements near a vessel wall so that the therapeutic elements are spaced around a perimeter of the vessel wall, and each of the radially expanded curved configurations tracing less than a 360 degree arc.

32. The system of clause 31, wherein each of the radially expanded curved configurations trace less than a 270 degree arc.

33. The system of clause 31, wherein each of the radially expanded curved configurations trace about a 180 degree arc.

34. The system of clause 31, wherein, when the plurality of expandable members are in the radially expanded curved configuration, at least part of each therapeutic element is intersected by a plane substantially orthogonal to a longitudinal axis of the elongated member.

35. The system of any one of clause 31 to 34, wherein each expandable member rotates about a corresponding axis in the radially expanded curved configuration, and wherein one or more axis is substantially parallel to a longitudinal axis of the elongated member.

36. The system of clause 35, wherein a first expandable member rotates in a different direction than a second expandable member in the radially expanded curved configurations.

37. The system of any one of clauses 31 to 36, wherein the plurality of expandable members are not connected at a distal portion of the plurality of expandable members.

38. The system of any one of clauses 31 to 37, wherein the plurality of expandable members consists of two expandable members.

39. The system of any one of clauses 13 to 38, further comprising a guide sheath.

40. The system of clause 39, wherein the plurality of expandable members are configured assume the radially expanded curved configuration when the plurality of expandable members extend from a distal port of the guide sheath.

41. The system of clause 39 or 40, wherein the guide sheath comprises an extended tapered tip configured to facilitate navigation of the guide sheath through the vasculature of the patient.

42. The system of any one of clauses 31 to 41, further comprising a guidewire, wherein one or more expandable members of the plurality of expandable members comprises an inner lumen, and wherein the guidewire is configured to be positioned within the inner lumen when the plurality of expandable members is in the delivery configuration.

43. The system of clause 42, wherein the plurality of expandable members are configured to transition from the delivery configuration to the radially expanded helical configuration in response to the guide member being withdrawn proximally from the one or more expandable members.

44. The system of any one of clauses 31 to 43, wherein each expandable member of the plurality of expandable members comprises a corresponding shape memory structure configured to urge the expandable member from the delivery configuration to the radially expanded curved configuration.

45. A method comprising:
navigating a catheter through vasculature of a patient to a target treatment site, wherein the catheter comprises:
   an elongated member; and
   a plurality of expandable members arranged at a distal portion of the elongated member, each expandable member comprising a corresponding one or more therapeutic elements arranged along a length of the expandable member;
expanding each expandable member of the plurality of expandable members from a delivery configuration to radially expanded curved configuration, wherein, when the plurality of expandable members are in the radially expanded curved configuration, the corresponding one or more therapeutic elements of each expandable member are positioned near a vessel wall so that the therapeutic elements are spaced around a perimeter of the vessel wall, and wherein each of the radially expanded curved configurations traces less than a 360 degree arc; and
delivering treatment to the vessel wall using the one or more therapeutic elements.

46. The method of clause 45, wherein, when the plurality of expandable members are in the radially expanded curved configuration, at least part of each therapeutic element is intersected by a plane substantially orthogonal to a longitudinal axis of the elongated member.

47. The method of clause 45 or 46, wherein each of the radially expanded curved configurations trace less than a 270 degree arc.

48. The method of clause 45 or 46, wherein each of the radially expanded curved configurations trace about a 180 degree arc.

49. The method of clause any one of clauses 45 to 48, wherein a first expandable member rotates in a different direction than a second expandable member in the radially expanded curved configurations.

50. The method of any one of clauses 45 to 49, wherein the plurality of expandable members are not connected at a distal portion of the plurality of expandable members.

51. The method of any one of clauses 45 to 50, wherein the plurality of expandable members consists of two expandable members.

52. The method of any one of clauses 45 to 51, wherein navigating the catheter through vasculature of the patient to the target treatment site comprises introducing the catheter through a guide sheath.

53. The method of clause 52, wherein expanding each expandable member of the plurality of expandable members comprises causing relative movement between the catheter and the guide sheath so that the plurality of expandable members extend from a distal port of the guide sheath.

54. The method of clause 51 or 53, wherein the guide sheath comprises an extended tapered tip configured to facilitate navigation of the guide sheath through the vasculature of the patient.

55. The method of any one of clauses 45 to 54, wherein navigating the catheter through vasculature of the patient to the target treatment site comprises advancing the catheter over a guidewire while the guidewire is positioned within an inner lumen of one of the expandable members while the plurality of expandable members are in the delivery configuration.

56. The method of clause 55, wherein expanding each expandable member of the plurality of expandable members comprises withdrawing the guide member proximally from the one or more expandable members.

57. The method of any one of clauses 45 to 56, wherein each expandable member of the plurality of expandable members comprises a corresponding shape memory structure, and wherein expanding each expandable member comprises urging the plurality of expandable members from the delivery configuration to the radially expanded curved configuration using the common shape memory structure.

1. A system comprising:
a catheter comprising:
an elongated member configured to be navigated through vasculature of a patient to a target treatment site; and
a plurality of expandable members arranged at a distal portion of the elongated member, each expandable member comprising a corresponding two or more therapeutic elements arranged along a length of the expandable member, each expandable member of the plurality of expandable members being configured to assume a delivery configuration and a radially expanded helical configuration, each expandable member being configured to position the corresponding two or more therapeutic elements near a vessel wall so that the therapeutic elements are spaced around a perimeter of the vessel wall, and wherein distal portions of the plurality of expandable members are connected to each other at a distal tip of the catheter.

2. The system of claim 1, wherein, when the plurality of expandable members are in the radially expanded helical configuration, at least part of each therapeutic element is intersected by a plane substantially orthogonal to a longitudinal axis of the elongated member.

3. The system of claim 1, wherein each expandable member of the plurality of expandable members is configured to, in the radially expanded helical configuration, rotate about a corresponding helical axis, and wherein one or more axes are substantially parallel to a longitudinal axis of the elongated member.

4. The system of claim 3, wherein the plurality of expandable members comprises a first expandable member and a second expendable member, and wherein when the first expandable member and the second expandable member are in the respective radially expanded helical configurations, the first expandable member rotates in a different direction than the second expandable member.

5. The system of claim 1, wherein the distal tip comprises an extended tapered tip configured to facilitate navigation of the elongated member and the plurality of expandable members through the vasculature of the patient.

6. The system of claim 1, wherein the plurality of expandable members consists of two expandable members.

7. The system of claim 1, further comprising a guide sheath.

8. The system of claim 7, wherein the plurality of expandable members are configured assume the radially expanded helical configuration when the plurality of expandable members extend from a distal port of the guide sheath.

9. The system of claim 7, wherein the guide sheath comprises an extended tapered tip configured to facilitate navigation of the guide sheath through the vasculature of the patient.

10. The system of claim 1, further comprising a guide member.

11. The system of claim 10, wherein the guide member comprises a guidewire, wherein one or more expandable members of the plurality of expandable members comprises an inner lumen, and wherein the guidewire is configured to be positioned within the inner lumen when the plurality of expandable members is in the delivery configuration.

12. The system of claim 10, wherein the plurality of expandable members are configured to transition from the delivery configuration to the radially expanded helical configuration in response to the guide member being withdrawn proximally from the one or more expandable members.

13. The system of claim 1, wherein the plurality of expandable members comprise a shape memory structure.

14. The system of claim 13, wherein the plurality of expandable members comprise a common shape memory structure, and wherein the shape memory structure is configured to urge the plurality of expandable members from the delivery configuration to the radially expanded helical configuration.

15. The system of claim 13, wherein each expandable member of the plurality of expandable members comprises a corresponding shape memory structure, and wherein the corresponding shape memory structures are configured to urge the corresponding expandable members from the delivery configuration to the radially expanded helical configuration.

16. A method comprising:
navigating a catheter through vasculature of a patient to a target treatment site, wherein the catheter comprises:
an elongated member; and
a plurality of expandable members arranged at a distal portion of the elongated member, each expandable member comprising a corresponding two or more therapeutic elements arranged along a length of the expandable member, and distal portions of the plurality of expandable members being connected to each other at a distal tip of the catheter;
expanding each expandable member of the plurality of expandable members from a delivery configuration to radially expanded helical configuration, wherein, when the plurality of expandable members are in the radially expanded helical configuration, the corresponding two or more therapeutic elements of each expandable member are positioned near a vessel wall so that the therapeutic elements are spaced around a perimeter of the vessel wall; and
delivering treatment to the vessel wall using the two or more therapeutic elements.

17. The method of claim 16, wherein, when the plurality of expandable members are in the radially expanded helical configuration, at least part of each therapeutic element is intersected by a plane substantially orthogonal to a longitudinal axis of the elongated member.

18. The method of claim 16, wherein each expandable member of the plurality of expandable members is configured to, in the radially expanded helical configuration, rotate about a corresponding helical axis, and wherein one or more of the helical axes are substantially parallel to a longitudinal axis of the elongated member.

19. The method of claim 16, wherein navigating the catheter through vasculature of the patient to the target treatment site comprises introducing the catheter through a guide sheath, and wherein expanding each expandable member of the plurality of expandable members comprises causing relative movement between the catheter and the guide sheath so that the plurality of expandable members extend from a distal port of the guide sheath.

20. The method of claim 16, wherein navigating the catheter through the vasculature of the patient to the target treatment site comprises advancing the catheter over a guide member while the guide member is positioned within an inner lumen of at least one expandable member of the plurality of expandable members, and wherein expanding each expandable member of the plurality of expandable members comprises withdrawing the guide member proximally from the inner lumen of the at least one expandable member.

21. A system comprising:
a catheter comprising:
an elongated member configured to be navigated through vasculature of a patient to a target treatment site; and
a plurality of expandable members arranged at a distal portion of the elongated member, each expandable member comprising a corresponding one or more therapeutic elements arranged along a length of the expandable member, each expandable member of the plurality of expandable members being configured to assume a delivery configuration and a radially expanded curved configuration, each expandable member being configured to, in the radially expanded curved configuration, position the corresponding one or more therapeutic elements near a vessel wall so that the therapeutic elements are spaced around a perimeter of the vessel wall, and each of the radially expanded curved configurations tracing less than a 360 degree arc.

22. The system of claim 21, wherein when the plurality of expandable members are in the radially expanded curved configuration, at least part of each therapeutic element is intersected by a plane substantially orthogonal to a longitudinal axis of the elongated member.

23. The system of claim 21, further comprising a guide member, wherein one or more expandable members of the plurality of expandable members comprises an inner lumen, wherein the guide member is configured to be positioned within the inner lumen when the plurality of expandable members is in the delivery configuration, and wherein the plurality of expandable members are configured to transition from the delivery configuration to the radially expanded helical configuration in response to the guide member being withdrawn proximally from the inner lumen.

24. The system of claim 21, further comprising a guide sheath, wherein the plurality of expandable members are configured assume the radially expanded helical configuration when the plurality of expandable members extend from a distal port of the guide sheath.
